(19) United States
(12) Patent Application Publication
(10) Pub. No.: US 2006/0212097 A1
(43) Pub. Date: Sep. 21, 2006

(54) Method and device for treatment of medical conditions and monitoring physical movements

(76) Inventors: Vijay Varadan, Fayette, AR (US); Robert Harbaugh, Hummelstown, PA (US)

Correspondence Address: MCQUAIDE BLASKO, 811 UNIVERSITY DRIVE, STATE COLLEGE, PA, 16801, US

(21) Appl. No.: 11/361,135

(22) Filed: Feb. 24, 2006

Related U.S. Application Data
(60) Provisional application No. 60/655,812, filed on Feb. 24, 2005.

(51) Int. Cl. A61N 1/00; A61B 5/103

(52) U.S. Cl. 607/62; 600/595; 607/2

(57) ABSTRACT

The subject invention utilizes MEMS devices and wireless data transmission means to monitor and sense certain patient conditions or reactions, such as changes in pressure, patient movements, and tremors. These sensor devices include but are not limited to MEMS gyroscopes, MEMS accelerometers, and MEMS pressure sensors. The data from the sensor means is then preferably wirelessly transmitted to a second MEMS device to treat or alter the medical condition that has been monitored.

### FIELD OF THE INVENTION

The present invention relates to the use of nanotechnology, MEMS devices and wireless data transmission means to monitor and treat physical activities, and medical and physiological conditions.

### BACKGROUND OF THE INVENTION

The subject invention utilizes MEMS devices and wireless data transmission means to monitor and sense certain patient conditions or reactions, such as changes in pressure, patient movements, and tremors. These sensor devices include but are not limited to MEMS gyroscopes, MEMS accelerometers, and MEMS pressure sensors. The data from the sensor means is then preferably wirelessly transmitted to a second MEMS device to treat or alter the medical condition that has been monitored. Although many of such individual devices have been previously disclosed and fabricated, their use specifically in conjunction with a wireless medical feedback, biofeedback, and treatment system and device is novel.

To date, companies have struggled with implementing wireless technologies into medical treatment modalities and devices. There have been significant drawbacks to such implementation, including the poor implantability of many silicon based technologies, inadequate means of converting and modulating frequencies generated by the wireless devices, and a lack of functional MEMS devices to be utilized in this fashion. The instant invention overcomes these problems. Although the invention overcomes problems associated with treatment of numerous medical and physiological conditions, several specific medical conditions are addressed in detail herein.

A. Current Drawbacks to Treatment for Parkinson's' Disease

Parkinson's disease is a progressive neurological disorder that results from the degeneration of neurons in a region of the brain that controls the movement of the nerve system. This degeneration creates a shortage of the brain signaling (neurotransmitter) known as dopamine, causing the movement impairments that characterize the disease. Dopamine is a chemical messenger responsible for transmitting signals between the substantia nigra and the next “relay station” of the brain, the corpus striatum, to produce smooth, purposeful muscle activity. Loss of dopamine causes the nerve cells of the striatum to fire out of control, leaving patients unable to direct or control their movements in a normal manner.

The four primary symptoms of Parkinson's disease are tremor or trembling in the hands, arms, legs, jaw and face; rigidity or stiffness of the limbs and trunk; bradykinesia or slowness of movement; and postural instability or impaired balance and coordination. Occasionally, the disease also causes depression, personality changes, dementia, sleep disturbances, speech impairments or sexual difficulties. The tremor is the major symptom for many patients, and it has a characteristic appearance. Typically, the tremor takes the form of a rhythmic back-and forth motion of the thumb and forefinger at three beats per second. This is sometimes called “pill rolling.” Tremor usually begins in a hand, although sometimes a foot or the jaw is affected first.

There is currently no cure for Parkinson's disease (PD). When the symptoms grow severe, doctors usually prescribe levodopa (L-dopa), which helps replace the brain's dopamine. L-dopa is a dopamine precursor, a substance that is transformed into dopamine by the brain. The prescription of high dosages of levodopa was the first breakthrough in the treatment of PD. Unfortunately, patients experience debilitating side effects, including severe nausea and vomiting. Sometimes doctors prescribe other drugs that affect doapmine levels in the brain. In patients that are severely affected, a kind of brain surgery known as pallidotomy has reportedly been effective in reducing symptoms. Pallidotomy is indicated for patients who have developed dyskinetic movements in reaction to their medications. It targets these unwanted movements, the globus pallidus, and uses an electrode to destroy the trouble- causing cells. Another type of brain surgery, in which healthy dopamine-producing tissue is transplanted into the brain, is also being tested.

The current treatment for PD employs deep brain stimulator electrodes to deliver continuous high-frequency electrical stimulation to the thalamus or other parts of the brain that control movement. These electrodes are implanted in the thalamus and connected to a pacemaker-like device in the chest, which the patient can switch on or off as symptoms dictate. High frequency stimulation of cells in these areas actually shuts them down, helping to rebalance control messages throughout the movement control centers in the brain. Deep brain stimulation (DBS) is useful for treating tremor, dyskinesias, and other key motor features of PD including bradykinesia and rigidity.

DBS requires a surgical procedure to place the electrode in the brain, connected by wire to a battery source. Electrode placement is performed under local anesthesia. The wire is implanted under the scalp and neck, and the battery is implanted in the chest wall just below the collar bone. A series of stimulation adjustments are required in the weeks following implantation. Frequently, the battery lasts for three to five years, and is replaced through an incision in the chest. This is typically done as an outpatient procedure. DBS is advantageous in that instead of destroying the overactive cells that cause symptoms in PD, it temporarily disables them by firing rapid pulses of electricity between four electrodes at the tip of the lead. A deep brain stimulator has three implantable components: a lead, an extension, and a neurostimulator. The lead is a thin, insulated coiled wire with four electrodes at the end that is implanted in the brain through a small opening in the skull. The extension is an insulated wire that is passed under the skin of the head, neck and shoulder to connect the lead to the neurostimulator. Finally, the neurostimulator is a battery-operated device that is implanted under the skin near the collarbone and generates electrical signals.

The drawbacks of this current technology include the following: (1) the hard wiring is known to disconnect and/or fracture during patient wear; (2) a battery replacement requires invasive surgery and thereby involves the risks attendant to surgery including infection, failure, and damage to surrounding tissue; (3) the battery life is limited, and therefore it is impractical to have the device operating at all times; and (4) the tremor motion of the specific part of the body is not sensed and controlled by DBS. These drawbacks limit the effectiveness of the current technology. There is, therefore, a need for a wireless microsystem comprising sensors that communicate with an implantable lead which in turn controls the frequency of electrical signals transmitted to electrodes of the lead.

In addition, there have been numerous recent advances in the miniaturization of medical devices. Devices employing nanotechnology and microelectromechanical systems (MEMS) can be fabricated at the molecular and millimeter levels, respectively. However, despite such advances, these technologies have yet to reach the implantable stage, primarily due to the numerous challenges encountered when implanting a device in the human body. One of the main limitations of implantable devices relates to the materials used for micromachining and fabricating MEMS. Well-established fabrication techniques employ silicon as a material for the implantable Microsystems. However, at neutral pH, silicon develops an oxide layer with surface silanol groups. These silanol groups ionize in water, resulting in a negative charge on the silicon surface which may promote biofouling. For instance, silicon implant studies have shown fibrosis and scar tissue formation. Such occurrences can limit the functioning of the implantable device. As a result, the clinical use of silicon-based microdevices has been limited due to the material's inability to effectively interface with biological systems. Accordingly, there is also a need for a non-immunogenic material that can be used in the fabrication of an implantable device.

### SUMMARY OF THE INVENTION

The present invention overcomes current shortcomings in technology, including the foregoing examples thereof, by providing a method and apparatus for wirelessly transmitting signals necessary for the treatment and monitoring of various medical conditions and physical activities. The invention enables healthcare providers to make critical assessments of medical conditions which were previously unattainable. It further enables one to accurately monitor a broad spectrum of physical activities. The method and apparatus described herein provide implantable accelerometers, gyroscopes and pressure sensor devices based on biocompatible materials. The present method and apparatus also employ novel software which enables sensors to effectively wirelessly transmit data generated from the monitoring of patient movements and conditions to a corresponding medical treatment device and to a physician.

### BRIEF DESCRIPTION OF THE DRAWINGS

FIG. 1 is a flow chart of object oriented software process control.

FIG. 2 is flow charge of the external sensor unit control.

FIG. 3 is an illustration of a micro-needle and tremor control device for use in patients having Parkinson's disease.

FIG. 4 depicts implantable, biocompatible apparatus and materials according to the present invention.

FIG. 5 is a high resolution TEM image of a carbon nanotube fabricated in accordance with the requirements of the present invention.

FIG. 6 is a schematic of the base antennae device utilized in the invention.

### DETAILED DESCRIPTION OF THE INVENTION

A. Overall System Architecture.

The present invention overcomes the shortcomings of the prior art by providing biocompatible materials for use in the microfabrication of implantable devices and systems. These biomolecular interfaces are also compatible with biological systems. The biocompatible materials disclosed herein are readily available, easily pattemable, compatible with the silicon process and less expensive than traditional materials. A water soluble, non-toxic and non-immunogenic polymer such as Poly(ethylene glycol)(PEG)/poly(ethylene oxide) (PEO) is a well-known polymer that can be used as a silicon coating for biological applications.

Silicon fabrication techniques can be used to prepare the devices. Similarly, materials compatible with biological systems (e.g. SU-8) can be synthesized. SU-8, an epoxy-based negative photoresist has properties that make it a useful economic alternative for producing polymeric microfluidic structures for several applications. The novel feature of SU-8 is that it is easy to functionalize with carbon Nanotubes, discussed below. The polymer forms a highly stable, chemically resistant polymeric structure after cross linking, which has a wide range of applications in bioMEMS. Its high aspect ratio features have been used to form structures for bioMEMS applications. Similarly, because it is ideal to construct composite materials with carbon nanotubes, it is the material of choice upon which to base implantable MEMS devices.

The present invention also overcomes the obstacles associated with creating wireless and implantable devices to monitor physical activities and medical conditions. The invention comprises a wireless microsystem including sensors that communicate with an implantable lead, which in turn controls the frequency of electrical signals transmitted to electrodes of the lead. The microsystem sensors wirelessly transmit detection of tremors directly to a thalamic deep brain stimulation unit. The unit is powered not through an implantable battery source, but through a battery source that is worn by the patient in the form of a wrist watch or other externally mounted source. The lithium batteries (3-5 volts) at the watch as well as at the hat module supplying dc power to the wireless devices. The transmitting power level is well within the FCC approved level of 5 mW for the wireless system.

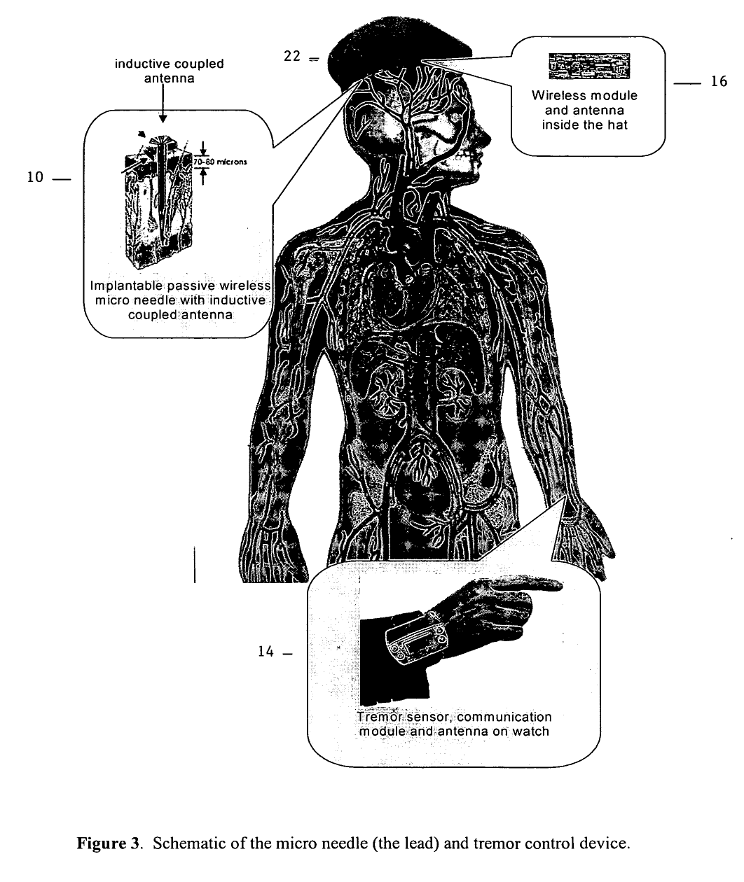

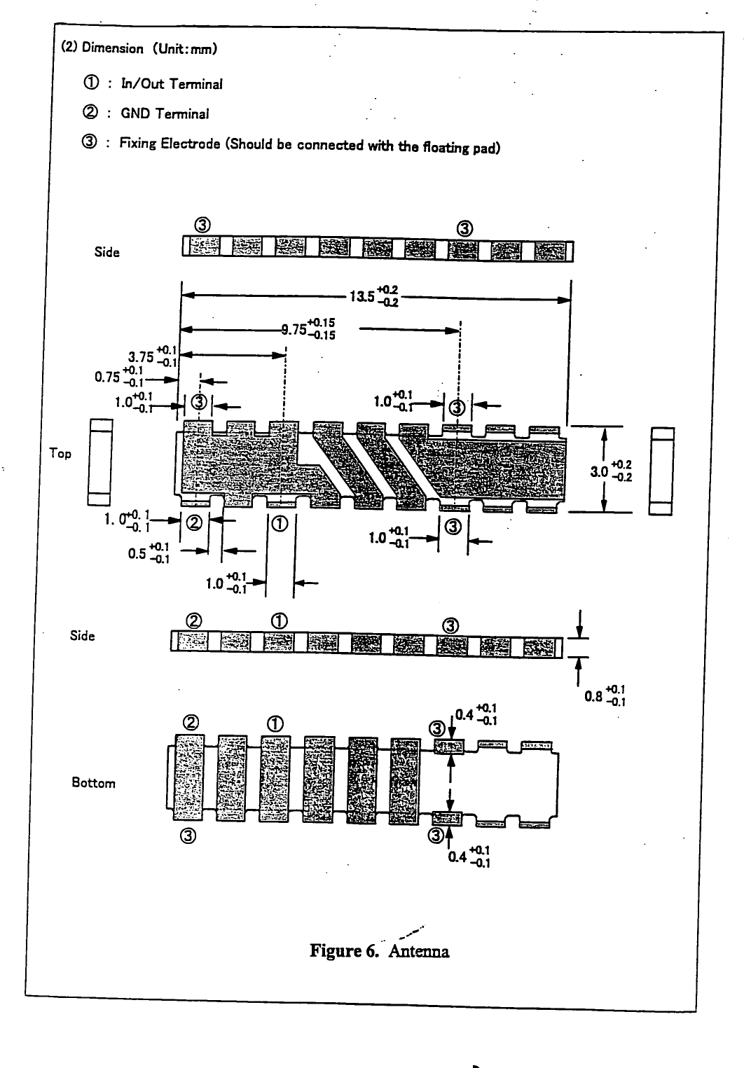

The wireless microsystem, depicted in FIG. 3, comprises a polymer MEMS based lead, 10, with an external wireless transceiver (located in a hat, wrist watch etc., 12), an accelerometer and gyro sensorunit, 14, for monitoring tremor motion, and a wireless control unit for monitoring and controlling tremor motion. The lead is preferably a polymer and carbon nanotube based system with a wireless transceiver. The micro-needles have a size on the order of human hairs and are easily implanted to the head. The implantable devices, which may be fabricated using shape-shifting polymers, are able to position very accurately inside the brain and can reposition by using thermal signals. The only component of the implantable device that is outside the skull is the inductive coupled antenna. The antenna, 16, is preferably approximately 4-6 mm and is attached to the micro-needle. The antenna architecture is shown in FIG. 6. It is preferably made of low temperature cofired ceramics (LTCC) with conventional integrated and embedded passive electronic components.

The miniaturization of many wireless and mobile communications equipment has been realized by the reduction of many electronic components (see e.g. Mitsubishi Materials Corporation; AHD1403-244ST01). This in-turn requires the reduction of antenna sizes. However, it is difficult to miniaturize many antennas without adversely impacting overall performances. Medical implants are intended to remain in the body for many years and are often necessary to communicate with control devices for the data transmission and reception. Thus, the design of antennas for miniaturized implantable devices is a challenging problem. These antennas should be small, compatible with the existing implantable devices and must be insulated from the body. In addition, close proximity of the human body needs to be addressed while designing these antennas. These antennas must not exceed the safety guidelines for power delivered to the body and should be insensitive to external EM noise.

One method of achieving very good antenna performance by miniaturization is to use high-permittivity multilayer ceramic substrates. These chip antennas, preferred because of their smaller sizes and lighter weights, could be able to adjust the resonance frequency by laser trimming. In multi-layer chip antennas the copper conducting patterns are embedded in the ceramic using LTCC technology. Ceramic substrates are made with mixing fine powders (for example BaO—Nd2O3-TiO2; BaO-(R203)y (TiO2)z.0.06(2Bi2O3.3TiO2)) of very small grain size with appropriate ratio. The antenna (multi-layer helical, spiral, Hilbert curve etc: depending on the impedance requirement of the wireless system) is patterned on to the substrate and then fired. Different layers of ceramic substrates are fabricated to achieve the desired impedance bandwidth. However thicker substrate can increase the bandwidth but will introduce large inductive reactance. Hence optimization of the substrate thickness is important for the final design. Although LTCC is very well suited for realizing RF and microwave components and antennas, many material properties are poorly characterized at RF frequencies and very little modeling data is available thereon. A Free Space Measurement system available from HVS Technologies, Inc., is a known method for such measurements, and this system may be utilized to optimize antenna performance for the instant invention.

Because the performance of an antenna depends mainly on the surrounding medium, it is necessary to use it close to the human body so that an efficient communication is possible within the small power (less than 5 mW). The specifications of the antennas are: 10% band width, gain 0-1 dB, with an operating temperature of −25 to +85° C. The antenna, 16, communicates with the external wireless module, 14, as shown in FIG. 3. The wireless module antenna, 16, is inductively coupled to the antenna on the micro-needle, 10, while at the same time it communicates with the antenna on the wireless module, 14, located on the arm, for sensing the tremor of the PD patient. When the sensor attached to the arm, 14, senses any tremor or vibration of the hand, it immediately communicates with the module located on the head, 12, and generates necessary electrical pulses. These pulses are transmitted to the micro-needle, 10, through the inductive coupled antenna for control of the tremor.

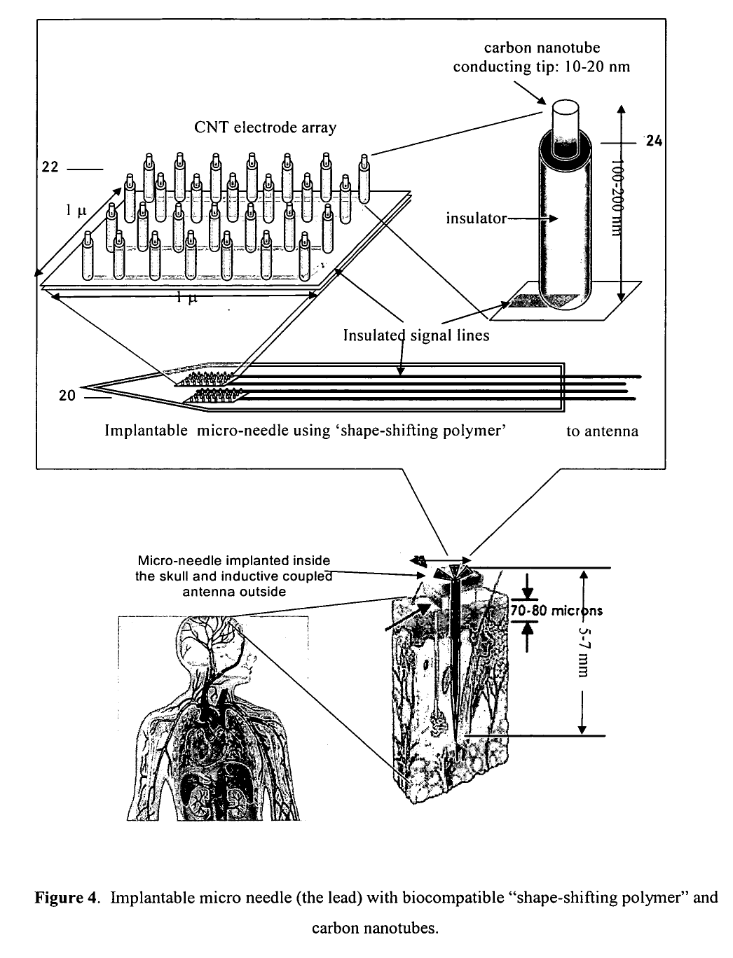

A diagram of the control system along with the micro-needle is shown in FIG. 4. The micro-needle, 20, includes an array of carbon nanotube conducting probe tips, 22, for delivering electrical pulses to the neuron. Arrays of these CNT conducting tips inside the insulator are electrically connected to the control electrode of the micro-needle using signal lines. The entire system fits inside the miniature implantable needle fabricated using shape-shifting materials.

B. MEMS Devices Utilized.

As used in this application, the term “Sensor” refers to a MEMS device that measures movement or change in pressure, and is preferably, but not necessarily, prepared using the functionalized carbon nanotube materials disclosed herein. The device can take the form of a MEMS accelerometer, MEMS gyroscope, MEMS pressure sensor, or similar device. The MEMS sensor of the instant invention provides advantages of light weight, small size, low power consumption and low cost, particularly when manufactured using standard integrated circuit fabrication techniques. A description as to the design and construction of a MEMS gyroscope is provided in U.S. Pat. No. 6,516,665, hereby incorporated into the present application by reference. Briefly, the gyroscope is fabricated as an integrated circuit using either a liftoff technique or a reactive ion etching technique. This device is similar to the MEMS accelerometer and pressure sensor utilized by the instant invention. A description of the MEMS accelerometer and pressure sensor technologies is contained in Varadan, V. K., Varadan, V. V. and Subramanian, H., Fabrication, characterization and testing of wireless MEMS-IDT based microaccelerometers, Sensors and Actuators A 90 (2001) 7-19. Regardless of the MEMS device used, the fabrication method includes the steps of providing a piezoelectric substrate having a surface, forming a pattern having a plurality of apertures therethrough, and fabricating, using the pattern, a plurality of features on the substrate. The features include resonator transducers, reflectors, a structure disposed on the surface, and sensor transducers separated from one another and disposed orthogonally to the pair of reflectors. A description of the carbon nanotube materials employed in said devices is contained in U.S. patent application No. 2004/0265212A1.

C. Carbon Nanotube Conducting Tip Array

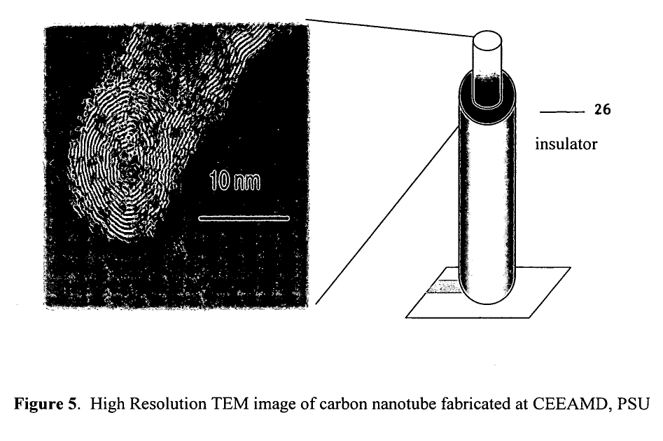

Micro-needles are commonly known for their advantages in medical applications. The conducting tip array of functionalized carbon nanotubes, 22 (fabricated by the CEEAMD group at The Pennsylvania State University) helps to reduce ohmic loss. Furthermore, the reduced size of the micro-needle produces minimal physical damage to living tissues while they are being implanted in the specimen and permits careful selection of the neural region to be triggered by the electrical pulses. The tip, 24, is preferably on the order of about 10-20 nm, also enables individual neurons to be selected. FIG. 5 depicts a high resolution TEM of the conducting tip, 26.

The present invention can be used to detect human motions, ranges of motion, tremors, pressure changes, brain electrical activity and similar medical or physiological conditions. This data is then wirelessly transmitted to a treatment modality or device, or to a data collection system. Because of the biocompatible materials utilized in the present sensors, the devices can be implanted or may be integrated into garments or articles of attire. The instant method and device can be used for a wide range of medical conditions, including Parkinson's disease, epilepsy, head injury, stroke, Alzheimer's disease, hydrocephalus and various physical therapy modalities.

Devices manufactured through use of the present carbon nanotube technology are lighter than steel and other conventional implantable technologies. In addition, the subject devices are exponentially stronger than existing steel technologies. Preferably, for several of the applications described herein, the biocompatible Sensor is implanted. The Sensors may also be embedded in articles of clothing, e.g. footwear or gloves, for monitoring physical therapy activity or for use in sporting and military applications. Significantly, the Sensors disclosed herein overcome the shortcomings of silicon based MEM devices, which are not suitable for implantation.

D. Software Utilized.

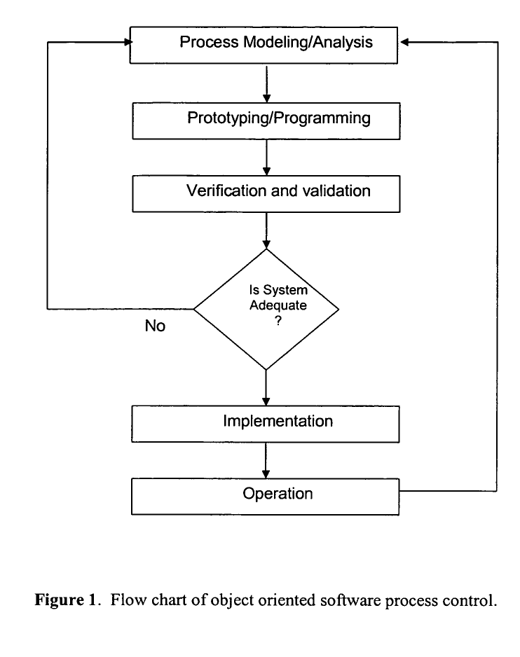

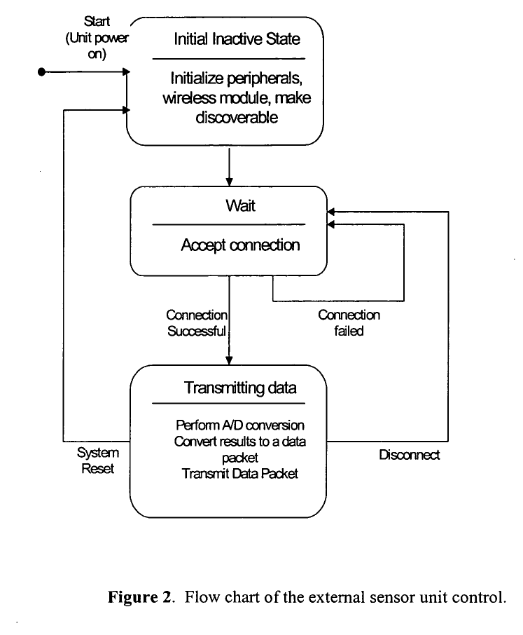

Controlling the gain of the antenna is a critical component to attaining a high functionality of the medical wireless systems as described herein. With the instant invention, the inventors have used software developed at and which may be obtained and licensed from The Pennsylvania State University as depicted in FIGS. 1 and 2, that dictates the pulse to be received by the antenna, and converts ordinary GHZ into a low frequency signal. The microcontrollers used in the “watch” control unit, as well as the receiving device are both Microchip PicMicro controllers which are RISC processors with built-in RAM and Flash ROM. Programs are written using Microchip Embedded C. The wireless module is connected to the microcontroller through the integrated serial communications (USART) port. It is controlled by the microcontroller which sends control commands and information to it in packets of digital data.

In the “watch” control unit, the microcontroller sends commands to generate the appropriate frequency for the specified duration. These commands are transmitted wirelessly to the implanted device as digital data over a 2.4 GHz digital wireless link established between the watch and receiving device. Connection management, data exchange and all other control functions are controlled by sending appropriate control commands to the wireless module.

At the receiving end, a microcontroller with software Pulse Width Modulation (PWM) capabilities is used to receive the commands from the watch and generate the required frequency in the electrodes. The frequency and duration of the pulse to be sent can be selected on the transmitting watch itself. Since this information is stored digitally, any frequency within the given range may be selected and transmitted.

For wireless communication, a wireless application protocol stack is developed and stored in both the sending and receiving devices. The use of data link management functions and error correction in the protocols ensures that the data is received as it was sent and minimizes packet loss. Thus it provides a high level of reliability. Using this protocol stack, data is sent at a maximum speed of 324 kbps which is adequate for the intended purpose. Different implanted devices can be identified for connection using the Physical Layer address unique to each device. This enables even an external doctor's computer to communicate with devices implanted in many patients and read data and control their operation.

This software allows for a more accurate and reliable method of wireless transmission of data previously unattainable with any known device. The software of the present invention has the architecture and features as set forth in FIG. 1.

E. Monitoring and Treatment of Medical Conditions.

As used herein, the term change in patient condition refers to a change in motion or motion patterns, or a change in fluid pressure.

1. Parkinson's Disease

In one embodiment of the invention, a MEMS gyroscope device is used to detect a patient's movements in extremities or other physical movements. As one example, a patient suffering from Parkinson's disease would exhibit tremors in the extremities that could be detected by the device. The wireless device, 14, would then transmit a signal to an implanted device in the brain, 10, designed to stimulate specific neurons. One configuration for such a system is depicted in FIG. 3. The present invention can advantageously be used to treat Parkinson's Disease. This involves implanting appropriate Sensors in the limbs of a patient with Parkinson's disease, enabling detection of tremors associated with Parkinson's disease. The Sensors wirelessly transmit data associated with such tremors directly to a thalamic deep brain stimulation unit. The unit is not powered by an implantable battery source, but by a battery source that can be worn by the patient in the form of a wrist watch or other externally mounted source.

In addition to deep brain stimulation, other treatment modalities for Parkinson's disease include injection of dopamine into the brain. Medical science has proven that Parkinson's disease occurs when the brain cells that produce dopamine die or fail to produce dopamine. Signs of Parkinson's tremors can also be detected by using the Sensors to wirelessly prompt a corresponding implanted device or pump to administer appropriate levels of dopamine.

In addition to treatment for Parkinson's disease, appropriate monitoring and feedback devices can be designed to monitor and treat a wide range of behavioral/neurological conditions, including obesity, obsessive compulsive disorder, and other specific neurological and psychiatric additions which may be treated by excitation of specific neurons in specific portions of the brain.

2. Intracranial Pressure In another embodiment of the invention, a MEMS pressure sensor may be employed to sense minute changes in pressure contained within a system or organ. For instance, intracranial pressures and intraventricular pressure may be wirelessly monitored in this fashion. Such wireless devices constitute a significant advance in medical monitoring. Current monitoring is invasive and carries certain surgical and post-surgical risks. With this invention, there would be no further need to tap the ventricular shunt.

Current technologies for measuring and monitoring intracranial pressure (ICP) require surgical implantation of a catheter that extrudes through the scalp and is connected to a strain gauge. Patients with such devices frequently have other traumatic injuries in addition to head injuries and must be transported through a hospital for various treatments. Current ICP monitoring technologies make patient transport difficult, and there is an attendant risk that the monitoring catheter will be dislodged with any movement of the patient or the external pressure monitor. This can impede health care providers from timely and efficiently providing necessary care to the patient. In addition, current technologies have a high risk of infection with prolonged use and therefore are not left in the patient for long periods of time. It is expected that the use of the present invention to monitor intracranial pressure will dramatically impact patient care by providing a simple and effective Sensor that eliminates the need for a monitoring catheter.

3. Hydrocephalus

Hydrocephalus occurs when cerebrospinal fluid (CSF) accumulates within the brain's ventricles or around the brain in the subarachnoid space. In patients with hydrocephalus, the CSF fails to be absorbed into the bloodstream and accumulates in the head. Current treatment modalities for hydrocephalus involve shunting CSF from the brain's ventricles, where an increase in pressure can cause injury. The most frequently employed treatment for hydrocephalus is currently the surgical placement of a ventriculo-peritoneal (VP) shunt. The shunt consists of a tube that is surgically inserted into the ventricles and connected to a tube under the scalp and skin leading to the abdomen where excess CSF is absorbed back into the body. A valve within the shunt regulates and prevents excess drainage.

Although VP shunts have been widely used for 30 years, they are associated with numerous complications such as infections, blockage, and eventual failure. Even the newly developed procedures for treatment of hydrocephalus have drawbacks. A significant drawback to current shunt technology, including flow and pressure regulated shunts and programmable shunts, is that they have minimal ability to regulate the CSF on a “real time” basis. For instance, the nature and degree of pressure depends upon the day to day and minute to minute activities of the patient. No current shunt technologies accommodate such real life conditions in regulating a shunt. The use of the present invention to monitor intracranial pressure and shunt flow rates, and/or to wirelessly control shunt function based specifically upon shunt and patient specific conditions, would dramatically improve shunt performance.

Endoscopic third ventriculostomy (ETV) uses special miniaturized tools and a small camera introduced through a tiny scalp incision to create an opening in the floor of the third ventricle. An alternative pathway of CSF flow is created around an obstruction in the usual pathway of CSF flow, allowing the CSF to be reabsorbed by the body. Although this minimally invasive surgery does not involve the implantation of any device in the body, it would be beneficial to be able to carefully monitor a patient's intracranial pressure following ETV to determine the effectiveness of the procedure in treating the obstruction to CSF flow. The present invention provides a fully implantable system for use in wireless monitoring of intracranial pressure. Accordingly, a patient's intracranial pressure can advantageously be monitored following ETV.

F. Monitoring Physical Movements.

1. Sporting Activities

Many sporting activities involve the accurate monitoring of physical motions. For instance, in the sport of golf, there are numerous devices developed to monitor and record one's golf swing. However, no current system allows a golfer's actual swing motions to be instantaneously recorded through a wireless, digital transmission of data. The Sensors of the present invention provide a new level of data analysis that has previously been unattainable. Similar applications can be envisioned in other sports.

2. Physical Therapy

Yet a further benefit to the current invention is that is allows for continuous monitoring both before and after treatment is administered through wireless transmission of data. For instance, in the case of a patient with Parkinson's disease, and a neuron stimulation device constructed with shape shifting polymers, physicians may monitor the effectiveness of the device both before and after different positions are employed in order to assess the efficacy of the device, and without any invasive procedure.

Yet a further embodiments of the invention involves a MEMS accelerometer device as disclosed in Varadan, V. K., Varadan, V. V. and Subramanian, H., Fabrication, characterization and testing of wireless MEMS-IDT based microaccelerometers, Sensors and Actuators A 90 (2001) 7-19. These devices may be used to monitor simple patient movements and could be employed to provide biofeedback in circumstances of gait retraining after stroke and general motor recovery treatment. Many such devices are cumbersome and include “hard wired” transmission systems which are inconvenient and limit patient movements. Use of the current inventions in these circumstances would provide virtually limitless patient freedom, as the MEMS devices are unobtrusive and would provide enhanced biofeedback.

Monitoring the actual range of human movement during physical therapy is also an application of the present invention. Such monitoring can be done not only during physical therapy sessions, but in a real world environment to determine specific activities for which restriction of movement is a problem. Further therapy can then be directed to these activities.

G. Additional Embodiments.

1. Protection from Interference.

Another embodiment of the invention involves encoding the transmission generated by each of the Sensors to employ its own individual identification number. Security is of utmost importance in such an application, to prevent devices from having unauthorized control over other devices, which can produce undesirable results. So, an RSA-based security algorithm is used to encrypt and control the wireless links between devices. This ensures proper operation of devices when more than one device is present in the same network. Also, for computers other than the user's watch to communicate with the implanted device, an appropriate security mechanism is used. In this fashion, various Sensors function despite potential sources of wireless transmission distortions, including interference from phone lines and other sources of transmission.

2. Use of Shape Shifting Polymers.

Current deep brain stimulus devices, including the device manufactured by Medtronics Inc., involves the use of a platinum electrode. This electrode may not be altered once it is surgically implanted.

It is well documented in the literature that currently available probes or devices to excite or stimulate neurons must be tediously and laboriously adjusted in the area of several millimeters within the brain in an attempt to maximize the placement and functionality of the device. Currently, this is done under surgery without meaningful radiological or imaging data. Once the device is surgically placed, there is no means to adjust that device absent further invasion surgery and exposure to anesthetics. One preferred embodiment of the current invention is to fabricate the needle device, 20, as depicted in FIG. 4, in part with shape shifting polymers. In this way, once the device is surgically implanted, it may be wirelessly and transcutaneously re-positioned through engaging the shape changing polymers.

Shape-shifting polymers are plastics that can alter their shape in response to temperature. These polymers have a memory that allows them to deform in temporary surroundings then return to their parent shape under suitable thermal stimulus. Shape- memory alloys such as nickel-titanium (Nitinol) have been used in actuators and medical devices. Even though these alloys are widely-used in medical applications, they have serious drawbacks. Primarily, they are able to achieve a maximum deformation of only about 8 percent, and they require high temperatures for programming. In contrast, the shape-shifting polymers of the present invention offer better deformation possibilities at lower temperature and have high shape stability. These shape-shifting polymers advantageously convert bulky implants into small devices that can be precisely positioned using endoscopes and then expanded to suit the surgical need. Although many formulations of polymers would be known to those skilled in the art, one such formulation is disclosed in this application. The disclosed formulation would be biocompatible for implant, and would also be compatible with electrodes manufactured from carbon nanotubes discussed above. The shape-shifting polymers of the present invention comprise two components with different thermal characteristics, namely, oligi(ε-caprolactone) diol and crystallisable oligo(ρ-dioxanone) diol. Both of these compounds are presently used in clinical applications. Shape shifting Polymers exhibit a radical change of shape from their normal state to a controlled state. The shape shifting can be done by external electric field as well as temperature. This change can be repeated without any degradation of the material. The “memory” comes from the stored mechanical energy attained during the application of the field.

The use of shape shifting polymers for the implantable device, 20, is helpful in maximizing accurate contact between the neurons of focus and the implantable devices because it could be possible to control the implantable electrodes using external circuits. No surgical procedure would be necessary to alter its position or neuron contact efficacy after the device is implanted.

While the invention has been particularly shown and described with reference to preferred embodiments thereof, it will be understood by those skilled in the art that various alterations in form and detail may be made therein without departing from the spirit and scope of the invention.

What is claimed is:

1. A system for treating medical conditions comprising:
(a) a first MEMS device for detecting a change in patient condition;
(b) an antenna means for wirelessly transmitting data generated by said first MEMS device; and
(c) a second MEMS device which is implanted in the patient in order to treat a medical condition based upon the data generated by the first MEMS device.

2. The system of claim 1 wherein the first MEMS device is a MEMS gyroscope.

3. The system of claim 1 wherein the second MEMS device includes electrodes comprising carbon nanotubes.

4. The system of claim 1 wherein the second MEMS device includes structural elements made from shape shifting polymers as a means of post surgically adjusting the placement of an electrode within brain tissue.

5. The system of claim 1 wherein the wireless transmission comprises an antenna made from low temperature cofired ceramics.

6. The system of claim 1 wherein the first MEMS device is a pressure sensor.

7. The system of claim 6 wherein the first MEMS device is manufactured using carbon nanotubes.

8. The system of claim 6 wherein the second MEMS device comprises a valve or shunt for regulating fluid pressure within a given system.

9. A system for monitoring and recording human physical movements comprising:
(a) a Sensor for detecting movement or pressure change; and
(b) a wireless transmission means for transmitting data from the Sensor to a recording or monitoring device.

10. The system of claim 1 wherein the first MEMS device is an accelerometer.

11. The system of claim 1 wherein the controller software dictates the pulse to be received by the antennae, and converts ordinary GHZ into a low frequency signal.
